(19) United States
(12) Patent Application Publication
(10) Pub. No.: US 2008/0305066 A1
(43) Pub. Date: Dec. 11, 2008

(54) 2-C-Substituted Propane-1,3-Dicarbonyl Compounds and Their Use in Neutralising Malodour

(75) Inventors: Felix Flachsmann, Duebendorf (CH); Jean-Pierre Bachmann, Wadenswil (CH)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 11/814,057

(22) PCT Filed: Jan. 6, 2006
(86) PCT No.: PCT/CH06/00010

§ 371 Date: Oct. 31, 2007

(30) Foreign Application Priority Data
Jan. 21, 2005 (GB) 0501237.2

(51) Int. Cl. A61L 9/00; C07C 69/587; C07C 69/618; A61K 31/22; C07C 69/604

(52) U.S. Cl. 424/76.1; 560/174; 560/201; 514/549; 560/176

(57) ABSTRACT

Use of a compound of formula 1 as malodour neutraliser

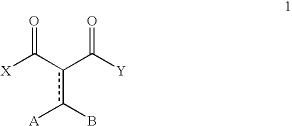

wherein A, B, Y and Y have the same meaning as given in the specification, and personal care products comprising the same.

The present invention refers to malodour neutralising compounds and to compositions containing them. More particularly, the present invention refers to the use of certain electrophilic alkenes and their hydroxyl- and alkoxy-adducts.

Malodours are offensive odours, which are encountered in the air and on many substrates such as fabrics, hard surfaces, skin, and hair. Malodours have either personal or environmental origin. For example sweat, urine and feces malodours are personal in origin, whereas kitchen and cooking malodours are of environmental origin.

While personal malodours are easily deposited on fabric, hair, and skin, environmental malodours also have a propensity to deposit on these substrates.

Amines, thiols, sulfides, short chain aliphatic and olefinic acids, e.g. fatty acids, are typical of the chemicals found in and contributed to sweat, household, and environmental malodours. These types of malodours typically include indole, skatole, and methanethiol found in toilet and animal odours; piperidine and morpholine found in urine; pyridine and triethyl amine found in kitchen and garbage odours; and short chain fatty acids, such as 3-methyl-3-hydroxyhexanoic acid, 3-methylhexanoic acid or 3-methyl-2-hexenoic acid, found in axilla malodours. Compounds which have been found in the axilla are described for example by Xiao-Nong Zeng et al., Journal of Chemical Ecology, Vol. 17, No. 7, 1991 page 1469-1492, which is incorporated herein by reference.

Several approaches have been used to counteract malodours. These approaches include masking by superimposing the malodour with a pleasant stronger odor, cross-adaptation by blocking of the malodour olfactory receptors, suppression of the malodour by mixing with an ingredient that causes a negative deviation according to Raoult's law, elimination of the malodour by absorption of the malodour by a porous or cage-like structure, and avoidance of the formation of malodours by such routes as antimicrobials and enzyme inhibitors. Although the methods known in the art have the ability to neutralize certain malodours, there still remains a need for further compounds which are even more efficient against malodours.

Surprisingly, the inventors now found a new class of compounds capable of neutralizing malodours.

Accordingly the present invention refers in a first aspect to the use as malodour neutraliser of a compound of formula 1

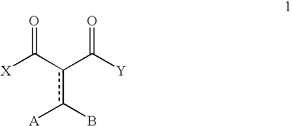

wherein
X and Y are independently a residue selected from the group consisting of —CR¹R²R³, wherein R¹, R², and R³ are independently H, a hydrocarbon residue, or a hydrocarbon residue containing at least one hetero atom selected from the list consisting of oxygen forming a hydroxyl-, carbonyl-, ether- or ester-group, nitrogen, silicon, chlorine and bromine;
—NR⁴R⁵, wherein R⁴ and R⁵ are independently H, a hydrocarbon residue, preferably C₁-C₂₀ alkyl, e.g. methyl, ethyl, propyl, butyl, isobutyl, 2-ethylhexyl, C₄-C₈ cycloalkyl, e.g. cyclopentyl and cyclohexyl; or R⁴ and R⁵ form together with the nitrogen atom to which they are attached a 3, 5 or 6-membered ring; or a hydrocarbon residue containing at least one hetero atom selected from the list consisting of oxygen forming a hydroxyl-, carbonyl-, ether- or ester-group, nitrogen, silicon, chlorine and bromine; and —OR⁶, wherein R⁶ is a hydrocarbon residue, preferably R⁶ is C₁-C₂₀ alkyl, e.g. methyl, ethyl, propyl, butyl, isobutyl, 2-ethylhexyl, tert.-butyl; C₃-C₈ cycloalkyl, e.g. cyclobutyl, cyclopentyl and cyclohexyl; C₃-C₁₀ alkenyl, e.g. propenyl, isopropenyl, and isobutenyl; C₆-C₁₀ aryl, e.g. phenyl or naphtyl; or C₇-C₁₀ alkylaryl, e.g. benzyl; or R⁶ is a hydrocarbon residue containing at least one hetero atom selected from the list consisting of oxygen forming a hydroxyl-, carbonyl-, ether- or ester-group, nitrogen, silicon, chlorine and bromine;
A is H, or —COOR⁷, wherein R⁷ is a hydrocarbon residue, preferably C₁-C₅ alkyl, e.g. methyl, ethyl, propyl, butyl, isobutyl, and tert.-butyl; C₂-C₅ alkenyl, e.g. propenyl, isopropenyl and isobutenyl; C₆-C₁₀ aryl, e.g. phenyl and naphtyl; C₇ to C₁₀ alkylaryl, e.g. benzyl; or a hydrocarbon residue containing at least one hetero atom selected from the group consisting of oxygen forming a hydroxyl-, carbonyl-, ether- or ester-group, nitrogen, silicon, chlorine and bromine;
—C(O)R⁸, wherein R⁸ is a hydrocarbon residue, preferably C₁-C₅ alkyl, e.g. methyl, ethyl, propyl, butyl, isobutyl, and tert.-butyl; C₃-C₅ alkenyl, e.g. propenyl, isopropenyl and isobutenyl, C₆-C₁₀ aryl, e.g. phenyl and naphtyl; C₇ to C₁₀ alkylaryl, e.g. benzyl; or a hydrocarbon residue containing at least one hetero atom selected from the group consisting of oxygen forming a hydroxyl-, carbonyl-, ether- or ester-group, nitrogen, silicon, chlorine and bromine; or
—CR⁹R¹⁰R¹¹, wherein R⁹, R¹⁰ and R¹¹ are independently H, a hydrocarbon residue, or a hydrocarbon residue containing at least one hetero atom selected from the group consisting of oxygen forming a hydroxyl-, carbonyl-, ether- or ester-group, nitrogen, silicon, chlorine and bromine;
B is H; —CR¹²R¹³R¹⁴, wherein R¹², R¹³ and R¹⁴ are independently H, a hydrocarbon residue; —OC(O)R¹⁸, wherein R¹⁸ is H, or a hydrocarbon residue, preferably C₁-C₂₀ alkyl, e.g. methyl, ethyl, propyl, butyl, isobutyl, 2-ethylhexyl, and tert.-butyl; C₃-C₈ cycloalkyl, e.g. cyclobutyl, cyclopentyl and cyclohexyl; C₃-C₁₀ alkenyl, e.g. propenyl, isopropenyl, and isobutenyl; C₆-C₁₀ aryl, e.g. phenyl or naphtyl; or C₇-C₁₀ alkylaryl, e.g. benzyl; or R¹², R¹³ and R¹⁴ are independently a hydrocarbon residue containing at least one hetero atom selected from the group consisting of oxygen forming a hydroxyl-, carbonyl-, ether- or ester-group, nitrogen, silicon, chlorine and bromine; and the dotted line together with the carbon-carbon bond represents a double bond; or
B is —NR¹⁶R¹⁷, wherein R¹⁶ and R¹⁷ are independently H, a hydrocarbon residue, preferably C₁-C₂₀ alkyl, e.g. methyl, ethyl, propyl, butyl, isobutyl, 2-ethylhexyl, and tert.-butyl; or C₃-C₈ cycloalkyl, e.g. cyclobutyl, cyclopentyl and cyclohexyl; or a hydrocarbon residue containing at least one hetero atom selected from the group consisting of oxygen forming a hydroxyl-, carbonyl-, ether- or ester-group, nitrogen, silicon, chlorine and bromine; or
—OR¹⁵, wherein R¹⁵ is H, a hydrocarbon residue, preferably C₁-C₂₀ alkyl, e.g. methyl, ethyl, propyl, butyl, isobutyl, 2-ethylhexyl, and tert.-butyl; C₃-C₈ cycloalkyl, e.g. cyclobutyl, cyclopentyl and cyclohexyl; C₃-C₁₀ alkenyl, e.g. propenyl, isopropenyl, and isobutenyl; C₆-C₁₀ aryl, e.g. phenyl or naphtyl; or C₇-C₁₀ alkylaryl, e.g. benzyl; or R¹⁵ is a hydrocarbon residue containing at least one hetero atom selected from the group consisting of oxygen forming a hydroxyl-, carbonyl-, ether- or ester-group, nitrogen, silicon, chlorine and bromine; and
the dotted line together with the carbon-carbon bond represents a single bond or a double bond;
with the proviso that A and B are not hydrogen at the same time.

Compounds of formula 1 wherein the dotted line together with the carbon-carbon bond represents a single bond also include all tautomeric forms thereof.

Particularly preferred are compounds of formula 1 wherein X or Y is a residue of the formula —CR¹R²R³ wherein R¹, R² and R³ form together with the carbon atom to which they are attached a residue selected from the group consisting of C₁-C₂₀ alkyl, e.g. methyl, ethyl, propyl, butyl, isobutyl, ethylhexyl and tert.-butyl, C₁-C₂₀ alkyl containing at least on hetero atom selected from the group consisting of O, N, Si, Cl, and Br, e.g. ethoxyethyl, methoxyethyl, 2-ethoxy-2-methylethyl, cyanomethyl, acetamidoethyl, diethylaminocarbamoylethyl and trimethylsilyloxylethyl;

C₃-C₈ cycloalkyl, e.g. cyclopentyl, cyclohexyl and cyclooctyl;
C₃-C₈ cycloalkyl containing at least one hetero atom selected from the group consisting of O forming a hydroxyl-, carbonyl-, ether- or ester-group, N, Si, Cl, and Br, e.g. oxacyclopropyl and tetrahydrofuranyl;
C₆-C₁₂ alkylcycloalkyl, e.g. methylcyclohexyl, ethylcyclohexyl, and methylcyclopentyl; C₆-C₁₂ alkylcycloalkyl containing at least one hetero atom selected from the group consisting of O forming a hydroxyl-, carbonyl-, ether- or ester-group, N, Si, Cl, and Br, e.g. (4-hydroxy)-cyclohexylmethyl;
C₆-C₁₂ cycloalkylalkyl, e.g. (4-methyl)-cyclohexyl and 2-(2-butyl)-cyclohexyl;
C₆-C₁₂ cycloalkylalkyl containing at least one hetero atom selected from the group consisting of 0 forming a hydroxyl-, carbonyl-, ether- or ester-group, N, Si, Cl, and Br, e.g. (4-methoxy)-cyclohexyl, (2-methoxy)-cyclohexyl and 4-azacyclohexyl;
C₂-C₁₀ alkenyl, e.g. propenyl, isopropenyl, isobutenyl;
C₂-C₁₀ alkenyl containing at least one hetero atom selected from the group consisting of forming a hydroxyl-, carbonyl-, ether- or ester-group, N, Si, Cl, and Br, e.g. ethyloxyvinyl and (2-propenyloxy)-ethyl;
C₂-C₁₀ alkinyl, e.g. propynyl;
C₂-C₁₀ alkinyl containing at least one hetero atom selected from the group consisting of forming a hydroxyl-, carbonyl-, ether- or ester-group, N, Si, Cl, and Br;
C₆-C₁₀ aryl, e.g. phenyl;
C₆-C₁₀ aryl substituted with at least one substituent selected from the group consisting of C₁-C₁₅ alkyl and C₁-C₁₅ alkoxy, e.g. p-methoxyphenyl;
C₅-C₁₀ heteroaryl, e.g. pyridinyl, furanyl, pyrryl, imidazolyl;
C₇-C₁₀ alkylaryl, e.g. benzyl; and
C₇-C₁₀ alkylaryl substituted with at least one substituent selected from the group consisting of C₁-C₁₅ alkyl, C₁-C₁₅ alkoxy, amino, C₁-C₁₅ alkylamino, and C₂-C₃₀ dialkylamino, e.g. methoxybenzyl, hydroxybenzyl, dihydroxybenzyl, (2-hydroxy-4-methoxy)-benzyl and (2-hydroxy-4-cyano)-benzyl.

Compounds of formula 1 wherein A is —CR⁹R¹⁰R¹¹ include compounds wherein R⁹, R¹⁰ and R¹¹ form together with the carbon atom to which they are attached a hydrocarbon residue selected from the group consisting of C₁-C₂₀ alkyl, e.g. methyl, ethyl, propyl, butyl, isobutyl, 2-ethylhexyl, and tert.-butyl; C₄-C₈ cycloalkyl, e.g. cyclopentyl and cyclohexyl; C₃-C₁₀ alkenyl, e.g. propenyl, isopropenyl, and isobutenyl; C₆-C₁₀ aryl, e.g. phenyl and naphtyl; C₆-C₁₀ aryl with at least one substituent selected from the group consisting of hydroxyl, C₁-C₅ alkoxy, phenoxy, benzyloxy, acetyl, benzoyl, amino, C₁-C₅ alkylamino, C₁-C₅ dialkylamino, nitro, chloro and bromo, e.g. (2-hydroxy-5-nitro)phenyl, (2-hydroxy-5-acetyl)phenyl, (2-hydroxy-4-methoxy)phenyl, (2-hydroxy)phenyl, and (3-ethoxy-4-hydroxy)phenyl; and C₅-C₈ heteroaryl, e.g. pyridinyl, furanyl, pyrryl, and imidazolyl.

Compounds of formula 1 wherein B is —CR¹²R¹³R¹⁴ include compounds wherein R¹², R¹³ and R¹⁴ form together with the carbon atom to which they are attached a hydrocarbon residue selected from the group consisting of C₁-C₁₀ alkyl, e.g. methyl, ethyl, propyl, butyl, isobutyl, 2-ethylhexyl, and tert.-butyl; C₄-C₈ cycloalkyl, e.g. cyclopentyl and cyclohexyl; C₃-C₁₀ alkenyl, e.g. propenyl, isopropenyl and isobutenyl; and C₆-C₁₀ aryl, e.g. phenyl or naphtyl.

Also preferred are compounds of formula 1 wherein A is —CR⁹R¹⁰R¹¹ and B is hydrogen or —CR¹²R¹³R¹⁴, and compounds of formula 1 wherein A is —COOR⁷ or —C(O)R⁸ and B is hydrogen or a residue selected from the group consisting of —CR¹²R¹³R¹⁴, —NR¹⁵R¹⁷, and —OR¹⁵.

Compounds of formula 1 wherein B is —NR¹⁶R¹⁷ and the sum of the carbon atoms of R¹⁶ plus R¹⁷ is greater than 10, are preferred.

Particularly preferred are compounds of formula 1 specifically described in Examples 1 to 34.

Also preferred are compounds of formula 1 selected from the group consisting of 2-(3,7-dimethyl-octa-2,6-dienylidene)-malonic acid diethyl ester; 3-octylidene-pentane-2,4-dione; 2-pyridin-2-ylmethylene-malonic acid diethyl ester; 2-octylidene-malonic acid dimethyl ester; 2-ethoxycarbonyl-but-2-enedioic acid diethyl ester; 2-acetyl-pent-2-enoic acid ethyl ester; 2-octylidene-malonic acid diethyl ester; 2-decylidene-malonic acid diethyl ester; 2-acetyl-dec-2-enoic acid ethyl ester; 2-(2-hydroxy-benzylidene)-1-phenyl-butane-1,3-dione; 2-(2-hydroxy-benzylidene)-malonic acid diethyl ester; 3-(2-hydroxy-benzylidene)-pentane-2,4-dione; 3-(2-hydroxy-4-methoxy-benzylidene)-pentane-2,4-dione; 2-(2-hydroxy-benzylidene)-3-oxo-butyric acid ethyl ester; 2-ethoxycarbonyl-oxymethylene-malonic acid diethyl ester; and 2-acetoxymethylene-malonic acid diethyl ester.

The inventors found that compounds according to the present invention are capable of neutralizing malodour compounds comprising a functional group selected from —SH, —NHR, or —NH₂ by chemical reaction with said group, thus neutralizing the malodour. Furthermore, the compounds of the present invention are capable to react with ammonia by chemical reaction. Whereas it is believed that the neutralization of malodour compounds by a compound of formula 1 wherein the dotted line together with the carbon-carbon bond represents a double bond occur via addition of the functional group to the electrophilic double bond, it is believed without to be bound by theory, that the neutralization of malodour compounds by a compound of formula 1 wherein the dotted line together with the carbon-carbon bond represents a single bond occur via an elimination/addition mechanism.

Surprisingly it was also found that certain compounds of formula 1 neutralize malodour compounds containing a carboxylic group, e.g. 3-methylhexanoic acid.

It was found that the compounds of formula 1 are much more active against malodour compounds, in particular in an aqueous environment, as for example dihexylfumarate (DHF), as is illustrated in the examples. Thus, a much lower concentration of a compound of the present invention is necessary to achieve a malodour reduction similar to the one obtained from DHF. Dihexylfumarate has already been used for a long time as a malodour counteractant and thus has been chosen as a comparison example.

By “active” is meant the reduction of a headspace concentration in % of a malodour compound. The headspace was analyzed by analysing a defined volume of the headspace of a test sample by GC-MS, as is described in more detail in the examples.

The compounds according to the present invention may be incorporated into a broad range of consumer products either by directly admixing the compound to the consumer product or by admixing a composition comprising a compound of formula 1, e.g. an alcoholic or aqueous solution containing further ingredients such as fragrances, which may then be mixed to the consumer product, using conventional techniques and methods. Thus, the invention additionally provides a method of manufacturing a composition comprising a compound of formula 1 as an active ingredient. It furthermore provides a method of manufacturing a consumer product comprising said compound as an active ingredient.

The amount of a compound of the present invention required for effective malodour neutralization depends upon the type of product into which such a compound is incorporated. It may further depend upon the ambient conditions, such as humidity and pH. For example, if used in a deodorant spray or room deodorizing spray, the product may comprise from about 0.01 to about 10% wt/wt of the final product, preferably from about 0.1 to about 1% wt/wt. If used in a room deodorizing filter device, i.e. a cooker hood, the amount of the compound may range from about 0.1% to about 20% wt/wt of the filter weight. Accordingly, the present invention refers in one of its aspects to a consumer product comprising about 0.01 to about 20 weight % of a compound of formula 1, or a mixture thereof.

A further aspect of the present invention is a method for imparting malodour neutralizing effects to a substrate, e.g. skin, hair or fabrics, comprising the step of contacting a substrate with a consumer product comprising a compound of formula 1.

The present invention also includes a process for dispersing a consumer product comprising a compound of formula 1 into a confined space, e.g. rooms, closets, chests, and draws. This process includes incorporating into a consumer product a compound of formula 1 and dispersing an effective amount of the consumer product into the space, e.g. by spraying, atomising and/or volatilising.

As used herein, “consumer products” include, for example cosmetic products, including products such as deodorants, antiperspirants, skin creams and lotions, shampoos, perfumes and toothpaste, and home care and fabric care products, including products such as air-fresheners, surface cleaners, detergents, fabric conditioners, rinsing conditioners for fabrics and products for application to garments, upholstery, curtains, carpets and absorbent materials.

Cosmetic products as used herein refers to articles intended to be applied to the human body for cleansing, beautifying, promoting attractiveness, or altering the appearance without affecting the body's structure of functions.

Absorbent materials include articles such as cat litter and filters. In a particular embodiment the term “filter” refers to any kind of filters used as part of a device to reduce air malodour for example in kitchens, ballrooms, litter bins, cars and refrigerators. Such devices include cooker hoods, vacuum cleaners and cat litter boxes,

The consumer product may be in form of a liquid, e.g. for application to a surface by pouring or spraying; a solid, e.g. a powder or compact powder, or in form of a candle; or a semisolid, such as a gel.

Whereas some compounds have been described in the literature, others have not, and are novel. Thus, in another aspect of the invention, there is provided a compound of formula 1a

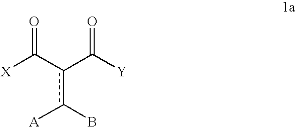

wherein
X and Y have the same definition as given above,
A is —COOR⁷, wherein R⁷ is a hydrocarbon residue, preferably C₁-C₅ alkyl, e.g. methyl, ethyl, propyl, butyl, isobutyl, and tert.-butyl; C₂-C₅ alkenyl, e.g. propenyl, isopropenyl and isobutenyl; C₆-C₁₀ aryl, e.g. phenyl and naphtyl; C₇ to C₁₀ alkylaryl, e.g. benzyl; or a hydrocarbon residue containing at least one hetero atom selected from the group consisting of oxygen forming a hydroxyl-, carbonyl-, ether- or ester-group, nitrogen, silicon, chlorine and bromine; or
A is —C(O)R⁸, wherein R⁸ is a hydrocarbon residue, preferably C₁-C₅ alkyl, e.g. methyl, ethyl, propyl, butyl, isobutyl, and tert.-butyl; C₃-C₅ alkenyl, e.g. propenyl, isopropenyl and isobutenyl, C₆-C₁₀ aryl, e.g. phenyl and naphtyl; C₇ to C₁₀ alkylaryl, e.g. benzyl; or a hydrocarbon residue containing at least one hetero atom selected from the group consisting of oxygen forming a hydroxyl-, carbonyl-, ether- or ester-group, nitrogen, silicon, chlorine and bromine;
B is —NR¹⁶R¹¹, wherein R¹⁶ and R¹⁷ are independently H, or a hydrocarbon residue; or
R¹⁶ and R¹⁷ are independently a hydrocarbon residue containing at least one hetero atom selected from the group consisting of oxygen forming a hydroxyl-, carbonyl-, ether- or ester-group, nitrogen, silicon, chlorine and bromine; or
B is —OR¹⁵, wherein R¹⁵ is H, or a hydrocarbon residue, preferably C₁-C₂₀ alkyl, e.g. methyl, ethyl, propyl, butyl, isobutyl, 2-ethylhexyl, and tert.-butyl; C₃-C₈ cycloalkyl, e.g. cyclobutyl, cyclopentyl and cyclohexyl; C₃-C₁₀ alkenyl, e.g. propenyl, isopropenyl, and isobutenyl; C₆-C₁₀ aryl, e.g. phenyl or naphtyl; or C₇-C₁₀ alkylaryl, e.g. benzyl; or
R¹⁵ is a hydrocarbon residue containing at least one hetero atom selected from the group consisting of oxygen forming a hydroxyl-, carbonyl-, ether- or ester-group, nitrogen, silicon, chlorine and bromine;
and the dotted line together with the carbon-carbon bond represents a single bond;
with the proviso that
if X═Y is ethoxy, B is not methoxy; and
if X is ethoxy and Y is methyl, B is not methoxy or ethoxy.

In particular embodiments are compounds of formula 1 a wherein A is —COOC₂H₅ and B is hydroxyl or —O—C₂H₅, for example, 3-acetyl-2-hydroxy-4-oxo-pentanoic acid ethyl ester; 2-acetyl-3-hydroxy-succinic acid diethyl ester; 2-benzoyl-3-hydroxy-succinic acid diethyl ester; 2-acetyl-3-hydroxy-succinic acid 4-ethyl ester 1-(2-methoxy-ethyl) ester; 3-benzoyl-2-ethoxy-4-oxo-pentanoic acid ethyl ester; 2-butyryl-3-ethoxy-succinic acid diethyl ester; and 2-benzoyl-3-ethoxy-succinic acid diethyl ester.

In a further aspect of the invention, there is provided a compound of formula 1b

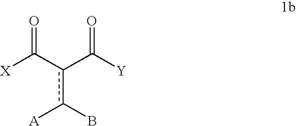

wherein
X and Y have the same definition as given above,
A is selected from the group consisting of n-heptyl, —COOC₂H₅, and —CH₂—CH(CH)₃— (CH₂)₂—CH═CH(CH₃)₂;
B is hydrogen;
and the dotted line together with the carbon-carbon bond represents a double bond;
with the proviso that
if X═Y is methyl or methoxy, A is not —COOC₂H₅;
if X═Y is methyl, methoxy or ethoxy, A is not n-heptyl;
if X is methyl and Y is ethoxy, A is not n-heptyl.

In particular embodiments are compounds of formula 1b selected from the group consisting of 2-benzoyl-dec-2-enoic acid ethyl ester; 2-acetyl-5,9-dimethyl-deca-2,8-dienoic acid ethyl ester; 2-acetyl-dec-2-enoic acid 2-(2-methoxy-ethoxy)-ethyl ester; 1-(4-tert-butyl-phenyl)-3-(4-methoxy-phenyl)-2-octylidene-propane-1,3-dione; 2-octylidene-malonic acid dibenzyl ester; 2-carbamoyl-dec-2-enoic acid methyl ester; 2-butyryl-but-2-enedioic acid diethyl ester; 2-acetyl-but-2-enedioic acid 1-benzyl ester 4-ethyl ester; 2-acetyl-but-2-enedioic acid 4-ethyl ester 1-(2-methoxy-ethyl) ester; 3-carbamoyl-4-oxo-pent-2-enoic acid ethyl ester; 2-(2,2-dimethyl-propionyl)-but-2-enedioic acid 4-ethyl ester 1-methyl ester; 2-pent-4-ynoyl-but-2-enedioic acid 4-ethyl ester 1-methyl ester; 4-ethyl 1-(2-(trimethylsilyl)ethyl)-2-acetylmaleate; ethyl-4-cyclohexyl-4-oxo-3-acetyl-but-2-enoate; ethyl 3-((trimethylsilyl)methylcarbamoyl)-4-oxopent-2-enoate; ethyl-4-(4-methylcyclohexyl)-4-oxo-3-acetyl-but-2-enoate; and 1-(2-(dimethylamino)ethyl) 4-ethyl 2-acetylmaleate.

Compounds of formula 1 wherein the dotted line together with the carbon-carbon bond represents a double bond, A represents —CR⁹R¹⁰R¹¹ and B is hydrogen may be prepared by condensation of an aldehyde of formula d with a dicarbonyl compound of formula e, known as the Knoevenagel condensation, as illustrated in scheme 1. The condensation may preferably be carried out in the presence of a small amount (typically 0.1-5 mol %) of a cyclic amine such as piperidine or pyrrolidine.

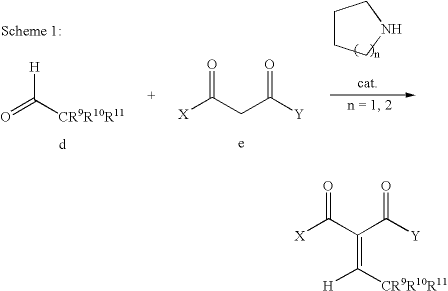

Compounds of formula 1 wherein the dotted line together with the carbon-carbon bond represents a double bond, A represents —CR⁹R¹⁰R¹¹ and B represents —CR¹²R¹³R¹⁴ may be prepared by condensation of a ketone of formula R⁹R¹⁰R¹¹C—C(O)—CR¹²R¹³R¹⁴ with a dicarbonyl compound of formula e in the presence of a Lewis acid such as TiCl₄, as described for example in Tetrahedron 1973, 29, 635-638 (W. Lehnert).

Similarly, compounds of formula 1 wherein the dotted line together with the carbon-carbon bond represents a double bond, A represents —COOR⁷ or —C(O)R⁸ and B represents —CR¹²R¹³R¹⁴ may be prepared by condensation of a ketoester of formula R⁷OOC—C(O)—CR¹²R¹³R¹⁴ or an α-diketone of formula R⁸(O)C—C(O)—CR¹²R¹³R¹⁴ with a dicarbonyl compound of formula e in the presence of a Lewis acid such as TiCl₄, as described for example in Tetrahedron 1972, 28, 663-666 (W. Lehnert).

Compounds of formula 1 wherein the dotted line together with the carbon-carbon bond represents a single bond, A represents —COOR⁷ and B is —OH may be prepared via addition of a dicarbonyl compound of formula e to a glyoxalate of formula d′ carried out preferably in the presence of a small amount (typically 0.1-5 mol %) of a cyclic amine such as piperidine or pyrrolidine, as illustrated in scheme 2.

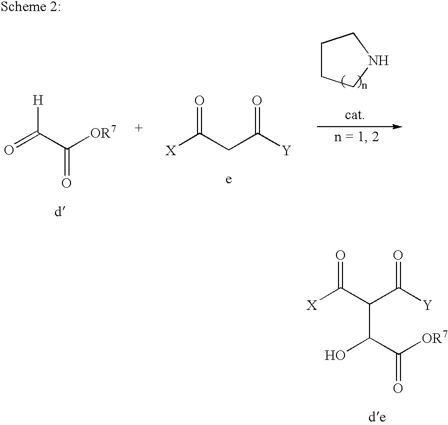

Similarly, compounds of formula 1 wherein the dotted line together with the carbon-carbon bond represents a single bond, A represents —C(O)R⁸ and B is —OH may be prepared via addition of a dicarbonyl compound of formula e to an alkylglyoxal of formula R⁸(O)C—CHO, as illustrated in scheme 3, carried out preferably in the presence of a small amount (typically 0.1-5 mol %) of a cyclic amine such as piperidine or pyrrolidine.

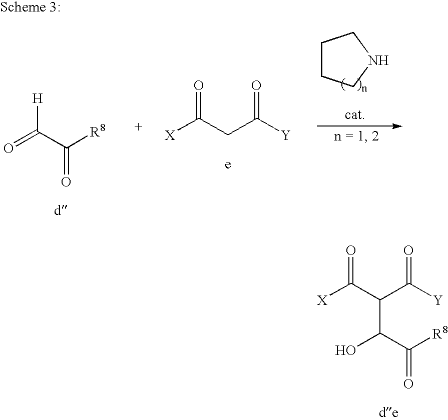

Compounds of formula 1 wherein the dotted line together with the carbon-carbon bond represents a double bond, A represents —COOR⁷ and B is H may be prepared starting from compounds of formula d′e by azeotropic removal of water in the presence of an acidic catalyst (typically 0.1-5 mol %) such as p-toluenesulfonic acid, using a water-azeotrope-forming organic solvent such as toluene or cyclohexane, as illustrated by scheme 4.

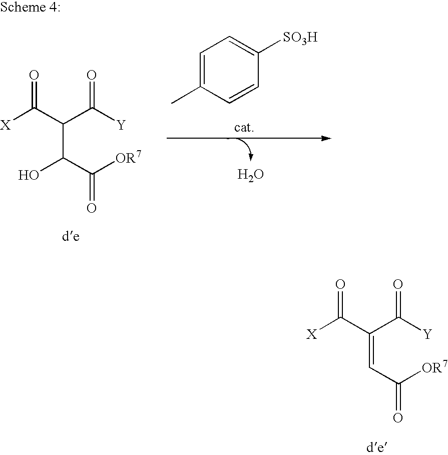

The addition step illustrated in scheme 2 and the dehydration step illustrated in scheme 3 may be performed successively in the same reaction vessel. In that case, the molar amount of acidic catalyst added for the dehydration step has to exceed the amount of cyclic amine catalyzing the addition step by at least 0.1 mol %.

Compounds of formula 1 wherein the dotted line together with the carbon-carbon bond represents a double bond, A represents —C(O)R⁸ and B is hydrogen may be prepared in a similar manner as described for the preparation of compounds of type d′e′.

Compounds of formula 1 wherein the dotted line together with the carbon-carbon bond represents a single bond, A represents —COOR⁷ and B represents —OR¹⁵ may be prepared by reaction of HOR¹⁵ to a compound of type d′e′, as illustrated in scheme 5, preferably at a temperature between 50-150° C. They may also be prepared by direct reaction of a compound of type d′e with HOR¹⁵. This reaction may be performed in the same reaction vessel as the reaction yielding d′e by simply adding an excess of HOR¹⁵ after the addition step is finished and heating up the resulting solution to 50-150° C.

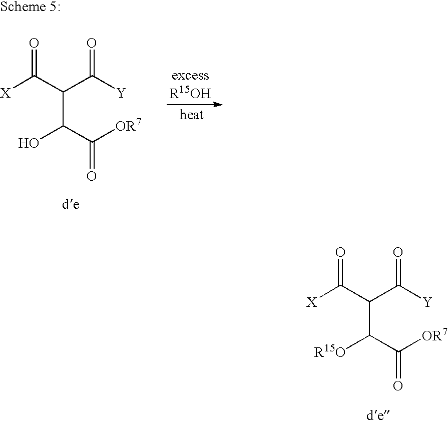

Compounds of formula 1 wherein B is —NR¹⁶R¹⁷ may be prepared by reaction of HNR¹⁶R¹⁷ similarly to the procedure described for the preparation of compounds of the type d′e″.

Compounds of formula 1 wherein the dotted line together with the carbon-carbon bond represents a double bond, A is H and B represents —OH or —OC(O)R¹⁸ can be prepared by reaction of a dicarbonyl compound of formula e with ethylformate or methylformate in the presence of a base, such as sodium methoxide, followed by acylation with an acyl chloride of the formula ClC(O)R¹⁸ as shown in Scheme 6.

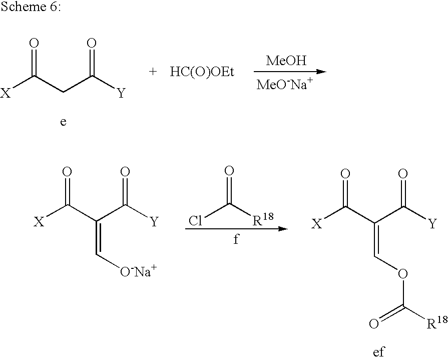

Similarly, compounds of formula 1 wherein the dotted line together with the carbon-carbon bond represents a double bond, A is —CR⁹R¹⁰R¹¹ and B represents —OH or —OC(O)R¹⁸ can be prepared according to the procedure for the preparation of compounds of type ef by using ClC(O)OC₂H₅.

The invention is now further described with reference to the following non-limiting examples.

These examples are for the purpose of illustration only and it is understood that variations and modifications can be made by one skilled in the art without departing from the scope of the invention. It should be understood that the embodiments described are not only in the alternative, but can be combined.

### EXAMPLE 1

### 2-Benzoyl-dec-2-enoic acid ethyl ester

Piperidine (0.10 ml, 0.5 mol %) is added to the mixture of octanal (25.6 g, 0.20 mol) and ethyl benzoylacetate (38.4 g, 0.20 mol) at 5° C. The resulting solution is warmed to room temperature and stirred for 24 h, during which a fine emulsion is formed. The mixture is diluted with methyl t-butyl ether and the organic layer washed with 2 N aq. HCl-solution, water and brine, then dried over MgSO₄.

The solvent is removed in vacuo and the residue distilled to yield 18.0 g (38%) of product as an E/Z-mixture, boiling at 129-135° C./0.1 mbar. The compound is an odourless oil.

¹³C-NMR (CHCl₃, 100 MHz; main isomer): 194.5 (s), 164.6 (s), 148.6 (d), 137.2 (s), 133.7 (d), 133.6 (s), 129.0 (d), 128.8 (d), 61.0 (t), 31.6 (t), 29.5 (t), 29.1 (t), 28.8 (t), 28.3 (t), 22.5 (t), 14. (q), 13.9 (q).

MS (EI, 70 eV): 302 (<1, M⁺), 257 (2), 217 (10), 199 (15), 186 (15), 171 (4), 157 (13), 105 (100).

### EXAMPLE 2-7

Following the general procedure of Example 1, the following compounds, all of which are colourless oils (unless otherwise stated) and essentially odourless, were prepared:

| | | | ¹³C-NMR | |
|---|---|---|---|---|
| | | Physical | CDCl₃, 100 MHz | MS |
| | Structure | data | (only main isomer) | El, 70 eV |
| 2 | 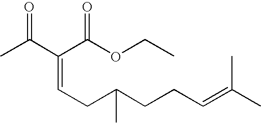 | b.p.127/0.07[° C./mbar] | 194.9 (s), 166.5 (s), 147.5 (d),139.8 (s), 131.6 (s), 124.2 (d),59.4 (t), 39.5 (t), 35.3 (t), 32.3 (d),27.4 (t), 25.7 (q), 22.4 (q), 19.6(q), 14.4 (q), 14.1 (q). | 266 (29, M⁺),221 (27), 205(26), 177 (29),149 (20), 43(100). |
| 3 | 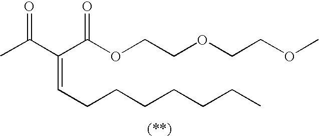 | b.p.168-171° C./0.08[° C./mbar] | 195.0 (s), 168.8 (s), 149.3 (d),136.6 (s), 71.8 (t), 70.4 (t), 68.9(t), 63.5 (t), 58.9 (q), 32.5 (t), 30.0(t), 29.2 (t), 29.0 (t), 27.0 (t), 22.5(t), 27.0 (q), 14.0 (q). | 272 (<1), 239(7), 195 (17),137 (45), 124(27), 59 (94), 45(100). |
| 4 | 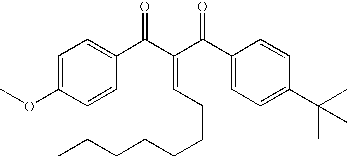 | * | 194.1 (s), 192.8 (s), 163.9 (s),156.2 (s), 147.9 (d), 141.9 (s),133.3 (s), 131.7 (d), 130.3 (s),129.5 (d), 125.4 (d), 123.7 (d),55.5 (q), 35.1 (s), 31.6 (t), 31.1(q), 31.0 (t), 31.0 (t), 29.2 (t), 28.9(t), 22.5 (t), 14.0 (q). | 420 (24, M⁺),363 (95), 335(39), 279 (36),255 (35), 161(91), 150 (97),135 (100). |
| 5 | 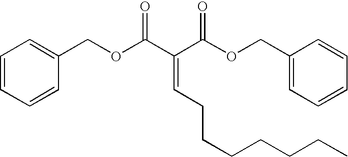 | * | 165.3 (s), 163.8 (s), 151.1 (d),135.6 (s), 135.4 (s), 128.5 (d),128.4 (d), 128.3 (d), 128.2 (d),128.0 (d), 128.0 (d), 67.0 (t), 66.9(t), 31.7 (t), 29.9 (t), 29.2 (t), 28.9(t), 28.3 (t), 22.6 (t), 14.1 (q). | 394 (5, M⁺), 200(16), 197 (20),179 (77), 123(15), 109 (12),91 (100). |
| 6 | 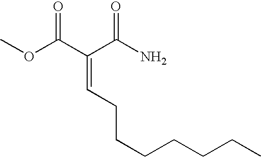 | * | 166.8 (s), 166.2 (s), 155.3 (d),127.3 (s), 52.3 (q), 31.6 (t), 30.4(t), 30.0 (t), 29.2 (t), 28.9 (t), 22.6(t), 14.0 (q). | 227 (3, M⁺), 210(19), 178 (39),168 (37), 153(62), 139 (65),113 (92), 81(93), 41 (100). |
| 7 | 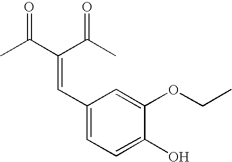 | *m.p.126-128° C.,white solid | 206.4 (s), 196.4 (s), 148.4 (s),146.0 (s), 140.6 (s), 140.1 (d),125.0 (s), 125.0 (d), 114.9 (d),112.2 (d), 64.6 (t), 31.7 (q), 26.3(q), 14.7 (q). | 248 (30, M⁺), 233(8), 2019 (4), 205(11), 191 (11),177 (11), 163(25), 145 (11), 43(100). |

(*) purified by chromatography
(**) The obtained product contains ca. 15% of 2-(1-Hydroxy-ethylidene)-dec-3-enoic acid 2-(2-methoxy-ethoxy)-ethyl ester. The starting material, 3-Oxo-butyric acid 2-(2-methoxy-ethoxy)-ethyl ester, is prepared as followed:

Ethyl acetoacetate (39.0 g, 0.30 mol) and diethyleneglycol monomethyl ether (36.0 g, 0.30 mol) are heated to 110° C. (oil bath temperature) and tetraisopropyl orthotitanate (0.60 ml, 2.0 mmol, 0.7 mol %) is added. The temperature is further increased to 150° C. After 30 min methanol (5 g) is distilling off and collected. The temperature is maintained for further 8 h while lowering the pressure in the apparatus to 800 mbar. After cooling to room temperature, the residue is distilled at 65-120° C./0.06 mbar to isolate 30.5 g of product containing ca. 10% of diethyleneglycol monomethyl ether (yield 44%).

¹³C-NMR (CHCl₃, 100 MHz): 200.4 (s), 167.0 (s), 89.9 (d), 71.8 (t), 70.4 (t), 68.8 (t), 64.2 (t), 59.0 (q), 50.0 (t), 30.0 (q).

MS (EI, 70 eV): 302 (<1, M⁺), 257 (2), 217 (10), 199 (15), 186 (15), 171 (4), 157 (13), 105 (100).

### EXAMPLE 8

### 2-Acetyl-3-hydroxy-succinic acid 4-ethyl ester 1-(2-methoxy-ethyl) ester

The mixture of ethyl glyoxylate (50% in toluene, 40.84 g, 0.20 mol) and 2-methoxy acetoacetate is cooled to 5° C. and treated with piperidine (0.10 ml, 1.0 mmol, 0.5 mol %). The mixture is slowly warmed to room temperature and stirred for 1 h. It is then diluted with methyl t-butyl ether, and the organic layer washed with 2N aq. HCl-solution and brine. After drying over MgSO₄, the solvents are removed in vacuo and the residue purified by flash chromatography on silica gel to yield 17.41 g of 2-Acetyl-3-hydroxy-succinic acid 4-ethyl ester 1-(2-methoxy-ethyl) ester as a colourless and odourless oil (33% yield). The product consists of a mixture of 2 diastereomers.

¹³C-NMR (CHCl₃, 100 MHz, both diastereomers): 201.7, 201.0 (s), 171.8, 171.8 (s), 167.6, 167.5 (s), 70.0, 69.9 (t), 69.7, 69.2 (d), 64.5, 64.4 (t), 62.1, 62.1 (t), 61.8, 61.7 (d), 58.8, 58.8 (q), 29.9, 29.8 (q), 13.9, 13.9 (q).

MS (EI, 70 eV): 263 (<1, [M+1]⁺), 245 (<1), 219 (6), 189 (36), 147 (45), 85 (39), 71 (43), 58 (65), 43 (100).

### EXAMPLE 9-14

Following the general procedure of Example 8, the following compounds, all of which are colourless oils and essentially odourless, were prepared:

| | | ¹³C-NMR | MS |
|---|---|---|---|
| | Structure | CDCl₃, 100 MHz | El, 70 eV |
| 9 | 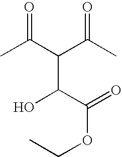 | (main isomere)203.2 (s), 202.2 (s), 171.9 (s), 69.5(d), 69.5 (d), 62.1, (t), 30.0 (q), 30.0(q), 13.9 (q). | — |
| 10 | 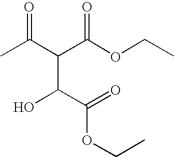 | 201.9 (s), 201.3 (s), 171.9 (2 s),167.6 (s), 167.5 (s), 69.9 (d), 69.2(d), 62.2 (2 t), 61.9 (2 t), 61.8 (d),61.6 (d), 30.0 (2 q), 14.0 (2 q). | 233 (<1,[M + 1]⁺), 159(3), 143 (3),130 (8), 117(20), 102 (6),88 (21), 85(19), 43 (100). |
| 11 | 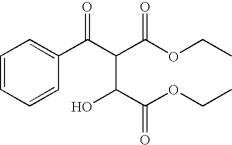 | 194.7 (s), 193.2 (s), 172.0 (s), 171.8(s), 167.7 (s), 167.4 (s), 136.2 (s),136.0 (s), 133.9 (d), 133.8 (d),128.8 (2 d), 128.5 (2 d), 70.6 (d),69.9 (d), 62.2 (t), 62.0 (t), 62.0 (t),61.9 (t), 56.8 (2 d), 14.0 (q), 13.9 (q). | 294 (<1, M⁺),276 (<1), 221(5), 192 (17),147 (7), 105(100). |
| 12 | 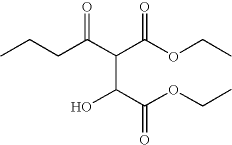 | 204.5 (s), 204.4 (s), 172.0 (2 s),167.7 (s), 167.5 (s), 70.0 (d), 69.3(d), 62.1 (t), 61.9 (t), 61.8 (t), 61.0(d), 60.9 (d), 44.7 (t), 44.6 (t), 16.7(2 t), 14.0 (q), 13.9 (q), 13.5 (q)13.4 (q). | 260 (<1, M⁺),201 (2), 190(8), 187 (21),173 (14), 143(16), 117 (35),71 (100). |
| 13 | 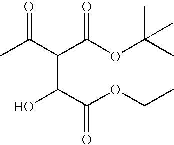 | 201.7 (s), 201.5 (s), 172.1 (2 s),166.7 (s), 166.5 (s), 83.2 (s), 83.0(s), 70.0 (d), 69.2 (d), 62.7 (d), 62.4(d), 62.1 (t), 30.0 (q), 29.9 (q), 27.8(q), 14.1 (q), 14.0 (q). | 261 (<1,[M + 1]⁺), 204(5), 187 (59),169 (5), 161(21), 131 (82),117 (66), 71(86), 57 (100). |
| 14 | 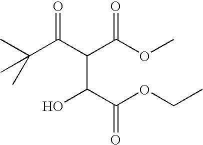 | 210.6 (s), 208.6 (s), 172.4 (s), 171.9(s), 167.6 (s), 167.3 (s), 71.3 (d),69.8 (d), 62.2 (t), 62.0 (t), 55.2 (d),55.0 (d), 52.8 (q), 52.5 (q), 45.4 (s),45.1 (s), 26.0 (q), 25.9 (q), 14.0 (q),13.9 (q). | 261 (<1,[M + 1]⁺), 203(3), 175 (8), 158(18), 130 (9),103 (21), 101(100), 69 (34),57 (77). |
| 15 | 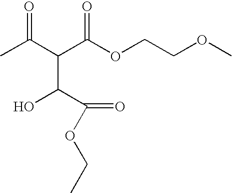 | 201.6 (s), 201.0 (s), 171.8 (2 s),167.5 (2 s), 69.7 (d), 69.2 (d), 61.8 (d),61.7 (d), 69.9 (2 t), 64.5 (t), 64.4 (t),62.1 (2 t), 58.8 (2 q), 29.9 (2 q),13.9 (2 q). | — |

### EXAMPLE 16

### 2-Butyryl-but-2-enedioic acid diethyl ester

The solution of 2-butyryl-3-hydroxy-succinic acid diethyl ester (10.0 g, 38.5 mmol) and p-toluenesulfonic acid (0.20 g) in cyclohexane is heated to reflux in a Dean-Stark apparatus during 3 h. The solution is cooled to room temperature, diluted with cyclohexane, and washed with water, dilute aqueous NaHCO₃-solution and brine. The organic layer is dried over MgSO₄ and concentrated in vacuo. The residue is bulb-to-bulb distilled at an oven temperature of 190-200° C. at 0.1 mbar to yield the product as colourless, essentially odourless oil (7.20 g, 77% yield).

¹³C-NMR (CHCl₃, 100 MHz): 202.2 (s), 195.9 (s), 165.5 (s), 164.1 (s), 164.0 (s), 162.9 (s), 147.2 (s), 144.5 (s), 128.4 (d), 127.7 (d), 62.3 (t), 62.0 (t), 61.7 (t), 61.7 (t), 44.6 (t), 41.2 (t), 16.9 (t), 16.3 (t), 14.0 (q), 13.9 (q), 13.9 (q), 13.5 (q), 13.5 (q).

MS (EI, 70 eV): 197 (11, [M-OEt]⁺), 169 (28), 143 (100), 125 (29), 71 (37).

### EXAMPLE 17-22

Following the general procedure of Example 16, the following compounds, all of which are colourless oils and essentially odourless, were prepared:

| | | ¹³C-NMR | MS |
|---|---|---|---|
| | Structure | CDCl₃, 100 MHz | El, 70 eV |
| 17 | 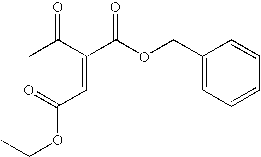 | 193.5 (s), 165.1 (s), 163.8(s), 144.1 (s), 134.8 (s),129.0 (d), 128.9 (d), 128.6(d), 128.5 (d), 67.8 (t),61.8 (t), 26.8 (q), 13.9 (q) | 277 (<1, [M + 1]⁺), 231(2), 170 (100), 142 (81),124 (68), 91 (85). |
| 18 | 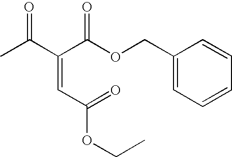 | 199.6 (s), 163.9 (s), 162.5(s), 146.6 (s), 134.5 (s),128.6 (2 d), 128.6 (d),128.1 (d), 67.8 (t), 61.7 (t),30.2 (q), 13.8 (q). | — |
| 19 | 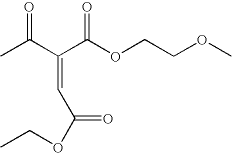 | (E and Z isomer)199.6 (s), 193.4 (s), 163.9(s), 162.6 (s), 144.1 (s),128.7 (d), 128.5 (d), 69.8(t), 69.8 (t), 65.0 (t), 64.7(t), 61.7 (t), 61.7 (t), 58.8(q), 58.7 (q), 30.1 (q), 26.7(q), 13.8 (q), 13.7 (q). | 245 (2, [M + 1]⁺), 229(6), 201 (13), 199 (6),170 (34), 167 (31), 142(30), 141 (55), 127 (19),124 (21), 99 (25), 58(100). |
| 20 | 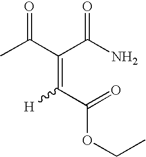 | (E and Z isomer)203.5 (s), 196.4 (s), 166.4(s), 164.8 (s), 164.5 (s),163.6 (s), 149.2 (s), 143.4(s), 131.2 (d), 126.1 (d),61.7 (t), 30.8 (q), 27.1 (q)13.9 (q), 13.8 (q). | 186 (6, [M + 1]⁺), 170(31), 156 (33), 140 (57),114 (53), 99 (45) 70(63), 43 (100). |
| 21 | 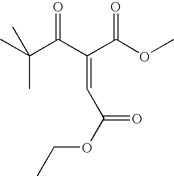 | (main isomer)203.8 (s), 165.0 (s), 164.0(s), 145.2 (s), 126.8 (d),61.6 (t), 52.8 (q), 45.1 (s),26.6 (q), 14.0 (q). | 186 (21, [M-C₄H₈]⁺),154 (100), 140 (35), 126(45). |
| 22 | 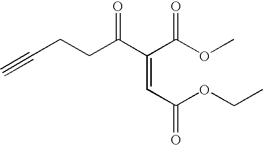 | 199.9 (s), 163.9 (s), 163.0(s), 146.1 (s), 129.3 (d),82.7 (s), 68.7 (d), 61.9 (t),53.1 (q), 41.5 (t), 13.9 (q),12.5 (t) | 238 (<1, M⁺), 185 (17),157 (76), 125 (32), 81(30), 53 (100). |

### EXAMPLE 23

### 2-Acetyl-3-methoxy-succinic acid diethyl ester

The solution of 3-acetyl-4-oxo-pent-2-enoic acid ethyl ester (3.0 g, 16.3 mmol), prepared according to procedure described by Example 14, in ethanol (20 ml) is heated to reflux for 20 h. The solvent is removed in vacuo and the residue purified by column chromatography on silical gel. The product is isolated as a colourless, essentially odourless oil (2.35 g, 63% yield).

¹³C-NMR (CHCl₃, 100 MHz; main isomer): 201.2 (s), 200.4 (s), 170.4 (s), 108.7 (s, enolform), 77.5 (d), 69.9 (d), 67.3 (t), 61.5 (t), 30.6 (q), 29.8 (q), 23.8 (q), 15.1 (q), 15.0 (q), 14.1 (q), 14.0 (e).

MS (EI, 70 eV): 197 (11, [M-OEt]⁺), 169 (28), 143 (100), 125 (29), 71 (37).

### EXAMPLE 24-27

Following the general procedure of Example 23, the following compounds, all of which are colourless oils and essentially odourless, were prepared:

| | | ¹³C-NMR | MS |
|---|---|---|---|
| | Structure | CDCl₃, 100 MHz | El, 70 eV |
| 24 | 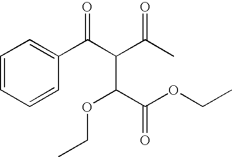 | 200.6 (s), 200.3 (s), 192.8 (s), 170.8 (s),170.3 (s), 137.1 (s), 136.1 (s), 133.8 (d),133.8 (d), 129.0 (d), 128.9 (d), 128.8 (d),128.8 (d), 128.7 (d), 78.3 (d), 78.3 (d),67.7 (t), 67.4 (t), 64.6 (d), 64.6 (d), 61.4(t), 61.4 (t), 29.5 (q), 29.4 (q), 15.1 (q),14.8 (q), 14.1 (q), 13.9 (q) | 246 (4, [M-EtOH]⁺), 219 (4),200 (6), 177 (14),131 (6), 105(100). |
| 25 | 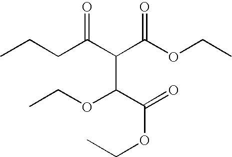 | 202.8 (s), 170.4 (s), 166.7 (s), 166.6 (s),77.4 (d), 77.3 (d), 67.6 (t), 67.5 (t), 61.8(t), 61.6 (t), 61.5 (t), 61.3 (t), 60.2 (t), 46.2(t), 44.4 (t), 16.7 (t), 16.6 (t), 15.1 (q), 15.0(q), 14.0 (q), 13.9 (q), 13.4 (q), 13.4 (q) | 242 (<1, [M-EtOH]⁺), 215 (2),196 (13), 171(13), 150 (13),145 (77), 143(36), 31 (100). |
| 26 | 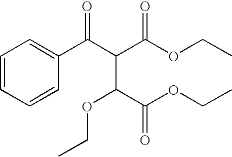 | 192.2 (s), 170.6 (s), 170.2 (s), 166.7 (s),136.9 (s), 133.8 (d), 133.5 (d), 128.9 (d),128.8 (d), 128.6 (d), 128.6 (d), 78.1 (d),77.8 (d), 67.8 (t), 67.7 (t), 61.8 (t), 61.7 (t),61.3 (t), 57.4 (d), 55.9 (d), 15.1 (q), 14.9(q), 14.1 (q), 14.0 (q), 13.9 (q), 13.8 (q) | 276 (2, [M-EtOH]⁺), 247 (3),231 (2), 105(100), 77 (22). |
| 27 | 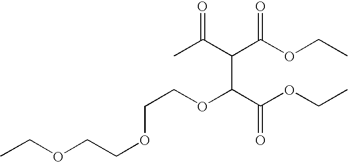 | 200.6 (s), 200.3 (s), 170.2 (s), 170.0 (s),166.6 (s), 77.7 (d), 71.5 (t), 71.2 (t), 70.5(t), 70.5 (t), 70.2 (t), 70.1 (t), 69.8 (t), 66.6(t), 62.1 (d), 61.7 (t), 61.6 (t), 61.4 (t), 61.1(t), 30.8 (q), 29.8 (q), 15.1 (q), 14.0 (q)13.9 (q). | 302 (<1, [M-EtOH]⁺), 259 (4),215 (6), 199 (3),187 (14), 109(24), 143 (24), 99(36), 73 (39), 59(40), 45 (100). |
| 28 | 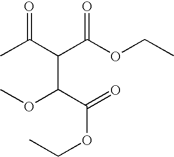 | 201.2 (s), 200.5 (s), 170.4 (s), 77.6 (d),69.9 (d), 67.3 (t), 61.5 (t), 30.6 (q), 29.8 (q),23.8 (q), 15.0 (2 q), 14.1 (q), 14.0 (q). | 184 (1, [M-EtOH]⁺), 169 (2),142 (13), 115 (40),87 (16), 43 (100). |

### EXAMPLE 29

### Neutralization of Propyl Mercaptane

A 23×75 mm-headspace vial is charged with 0.25 ml of a 4 mM-solution of the test compound in dimethylsulfoxide/H₂O 1:1. One blank sample containing 0.25 ml of dimethylsulfoxide/H₂O 1:1 is prepared per 3 test samples. The vials are sealed with a 20 mm-aluminium seal containing a rubber septum. At 15 min-intervals (which is the time of 1 GC-analysis run) 0.25 ml of a 4 mM-solution of propyl mercaptane in dimethylsulfoxide/H₂O 1:1 is added to each sample via cannula.

The samples are left at room temperature for 3 h, then submitted to headspace analysis using a headspace auto sampler connected to a GC-MS apparatus. Per sample 250 μl of headspace is injected. The peak areas (MS-ion current) of propyl mercaptane is compared to the averaged value from the blank samples to calculate the reduction of headspace concentration. The results are listed in Table 1 below.

TABLE 1
Headspace reduction

| | % Headspace | | % Headspace |
|---|---|---|---|
| Test Compound | Reduction | Test Compound | Reduction |
| DHF (Dihexylfumarate)comparison example | 5 | 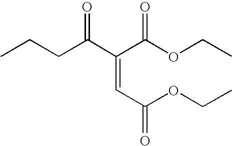 | 100 |
| 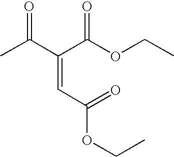 | 100 | 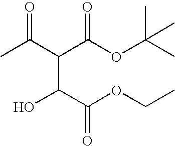 | 45 |
| 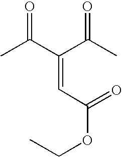 | 100 | 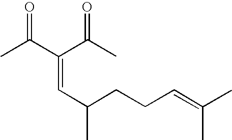 | 8 |
| 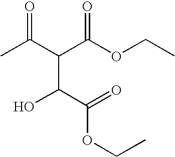 | 62 | 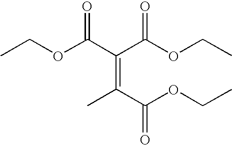 | 20 |
| 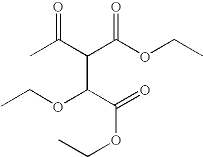 | 64 | 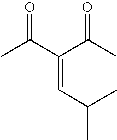 | 34 |
| 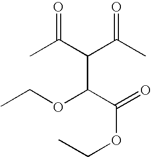 | 82 | 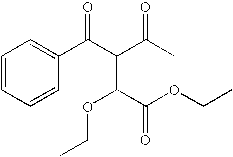 | 22 |
| 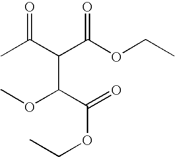 | 54 | 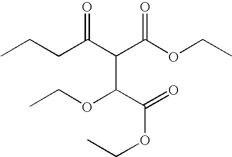 | 38 |
| 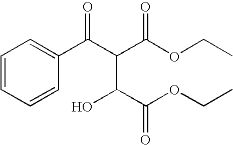 | 20 | 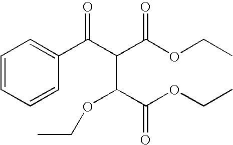 | 10 |
| 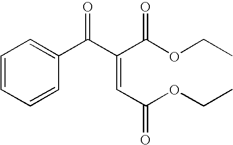 | 100 | 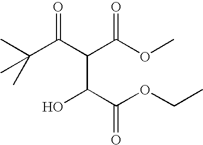 | 15 |
| 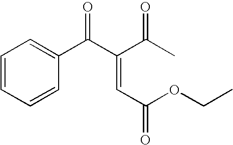 | 100 | 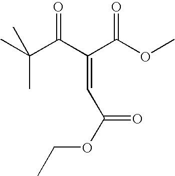 | 82 |
| 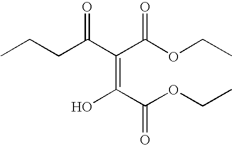 | 15 | | |

### EXAMPLE 30

### Neutralization of Human Sweat in Aqueous Environment

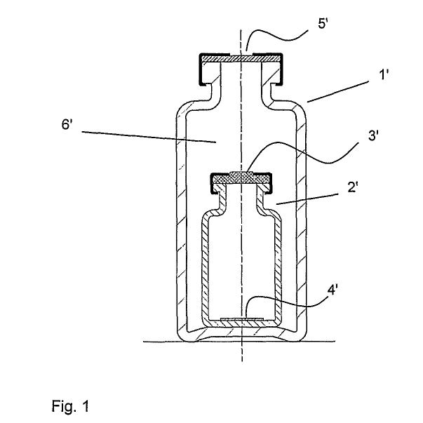

A two chamber sample was prepared as depicted in FIG. 1. It is composed of the headspace vial (1′) described in example 26 as the outer containment and a standard HPLC-auto sampler vial (2′) as the inner containment. The test compound (5.0 μmol) is absorbed on viscose filter (3′) (applied as CH₂Cl₂-solution and let evaporate). An amount of 100 ul of an aqueous solution of 3-methyl hexanoic acid (23 mM) and 3-mercapto-1-butanol (17 mM), is placed on the bottom of the inner vial (2′). A screw cap containing the filter (3′) is screwed tightly on the HPLC-vial (2′), which is placed in the headspace vial (1′). The headspace vial is closed with an aluminium seal containing a rubber septum (5′). Blank samples containing an empty filter (3′) and the aqueous solution of 3-methyl hexanoic acid and 3-mercapto-1-butanol in the inner vial as described above are prepared (1 blank per 3 test samples). The samples are left standing for 16 h at 35° C., then submitted to headspace analysis as described in example 26.

The malodourant compounds (3-methyl hexanoic acid and 3-mercapto-1-butanol) diffuse through the filter into the outer volume (6′). Therefore a compound of formula 1 (neutralizing substance) applied on the filter (3′) will reduce the headspace concentration of the sweat malodourants in the outer volume compared to the blank samples. The headspace reductions observed for several compounds of the present invention are listed in Table 2.

TABLE 2
% Headspace reduction of malodours compounds.

| Test compound | 3-mercapto-1-butanol | 3-methyl hexanoic acid |
|---|---|---|
| DHF | 10 | 0 |
| comparison | | |
| example | | |
| 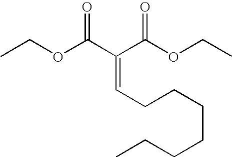 | 22 | 0 |
| 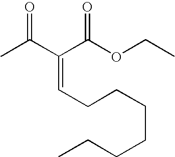 | 35 | 10 |
| 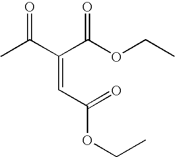 | 98 | 10 |
| 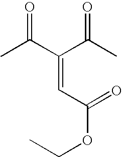 | 100 | 0 |
| 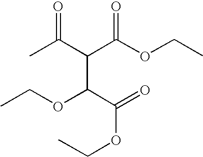 | 100 | 0 |
| 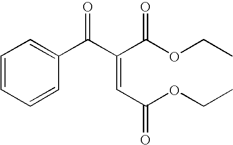 | 85 | 28 |
| 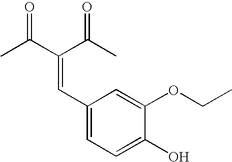 | 41 | 9 |

### EXAMPLE 31

### Olfactory Evaluation Against Sweat-Malodour Reconstitution

An ethanolic solution of axilla-malodour reconstitution is applied on a 1×1 cm cotton swatch (100 ∥l, 0.1% wt/wt), then 50 μl of the test compound (1% solution in ethanol) are dosed to the malodour treated swatches. The malodour intensity is rated on a LMS (labelled magnitude scale) by an expert panel of 20 assessors. The results are then expressed in % reduction relative to malodour of a swatch without adding a test compound. The results are shown in Table 3.

TABLE 3
Reduction of axilla malodour intensity in panel test.

| Test compound | % Reduction |
|---|---|
| 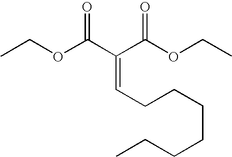 | 59 |
| 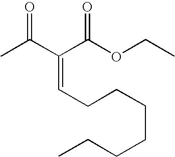 | 82 |
| 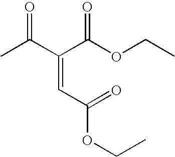 | 76 |
| 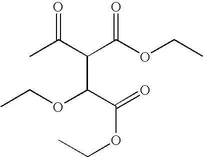 | 67 |
| DHF | 68 |
| comparison example | |

The experiment shows that most compounds are perceivably more efficient or at least similarly efficient in reducing axilla malodour than dihexylfumarate (DHF).

### EXAMPLE 32

The following compounds of the present invention may also be prepared according to the general procedure of Example 1:

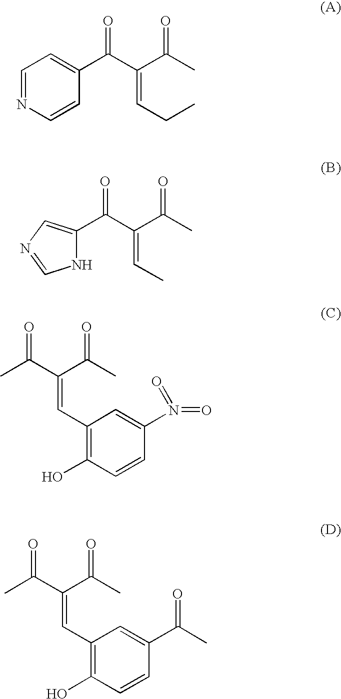

### EXAMPLE 33

The following compounds of the present invention may also be prepared according to the general procedure of Example 8:

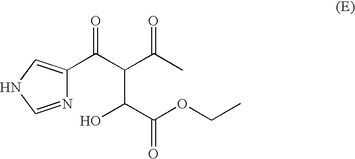

### EXAMPLE 34

The following compounds of the present invention may also be prepared according to the general procedure of Example 15:

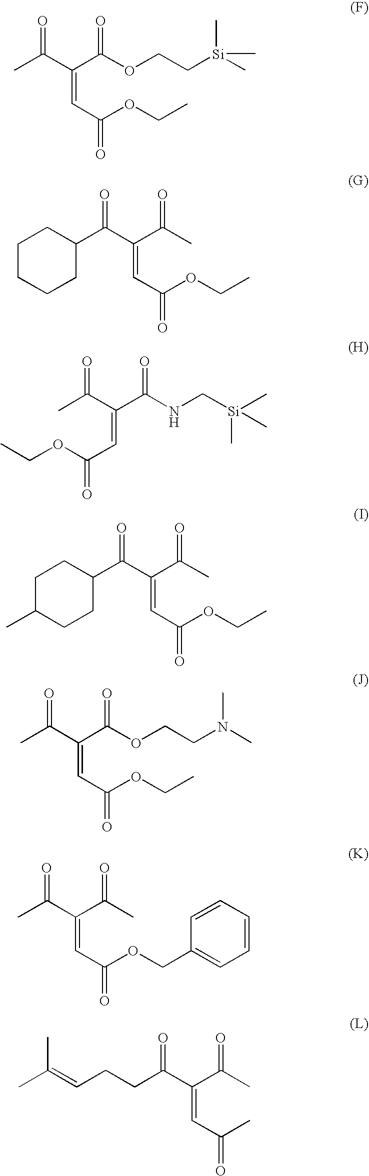

1. A method of neutralizing malodours comprising the use of a compound of formula 1 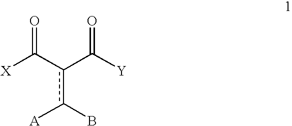
wherein
X and Y are independently a residue selected from the group consisting of —CR¹R²R³, wherein
R¹, R², and R³ are independently H, a hydrocarbon residue, or a hydrocarbon residue containing at least one hetero atom selected from the list consisting of oxygen forming a hydroxyl-, carbonyl-, ether- or ester-group, nitrogen, silicon, chlorine and bromine;
—NR⁴R⁵, wherein
R⁴ and R⁵ are independently H, a hydrocarbon residue; or
R⁴ and R⁶ form together with the nitrogen atom to which they are attached a 3, 5 or 6-membered ring; or R⁴ and R⁵ are a hydrocarbon residue containing at least one hetero atom selected from the list consisting of oxygen forming a hydroxyl-, carbonyl-, ether- or ester-group, nitrogen, silicon, chlorine and bromine;
and
—OR⁶, wherein
R⁶ is a hydrocarbon residue; or
R⁶ is a hydrocarbon residue containing at least one hetero atom selected from the list consisting of oxygen forming a hydroxyl-, carbonyl-, ether- or ester-group, nitrogen, silicon, chlorine and bromine;
A is H; —COOR⁷, wherein
R⁷ is a hydrocarbon residue; or
R⁷ is a hydrocarbon residue containing at least one hetero atom selected from the group consisting of oxygen forming a hydroxyl-, carbonyl-, ether- or ester-group, nitrogen, silicon, chlorine and bromine;
—C(O)R⁸, wherein
R⁸ is a hydrocarbon residue; or
R⁸ is a hydrocarbon residue containing at least one hetero atom selected from the group consisting of oxygen forming a hydroxyl-, carbonyl-, ether- or ester-group, nitrogen, silicon, chlorine and bromine;
or
—CR⁹R¹⁰R¹¹, wherein
R⁹, R¹⁰ and R¹¹ are independently H, a hydrocarbon residue, or a hydrocarbon residue containing at least one hetero atom selected from the group consisting of oxygen forming a hydroxyl-, carbonyl-, ether- or ester-group, nitrogen, silicon, chlorine and bromine;
B is H; —CR¹²R¹³R¹⁴, wherein
R¹², R¹³ and R¹⁴ are independently H, or a hydrocarbon residue; or
R¹², R¹³ and R¹⁴ are independently a hydrocarbon residue containing at least one hetero atom selected from the group consisting of oxygen forming a hydroxyl-, carbonyl-, ether- or ester-group, nitrogen, silicon, chlorine and bromine;
or
—OC(O)R¹⁸, wherein
R¹⁸ is H, or a hydrocarbon residue;
and the dotted line together with the carbon-carbon bond represents a double bond; or
B is —NR¹⁶R¹⁷ wherein
R¹⁶ and R¹⁷ are independently H, or a hydrocarbon residue; or
R¹⁶ and R¹⁷ are independently a hydrocarbon residue containing at least one hetero atom selected from the group consisting of oxygen forming a hydroxyl-, carbonyl-, ether- or ester-group, nitrogen, silicon, chlorine and bromine; or
—OR¹⁵, wherein
R¹⁵ is H, or a hydrocarbon residue; or
R¹⁵ is a hydrocarbon residue containing at least one hetero atom selected from the group consisting of oxygen forming a hydroxyl-, carbonyl-, ether- or ester-group, nitrogen, silicon, chlorine and bromine;
and the dotted line together with the carbon-carbon bond represents a single bond or a double bond;
with the proviso that A and B are not hydrogen at the same time.

2. The method according to claim 1 wherein in the a compound of formula 1:
X is the residue of the formula —CR¹R²R³,
R¹, R² and R³ form together with the carbon atom to which they are attached a residue selected from the group consisting of:
C₁-C₂₀ alkyl;
C₁-C₂₀ alkyl containing at least on hetero atom selected from the group consisting of O, N, Si, Cl, and Br;
C₃-C₈ cycloalkyl;
C₃-C₈ cycloalkyl containing at least one hetero atom selected from the group consisting of 0 forming a hydroxyl-, carbonyl-, ether- or ester-group, N, Si, Cl, and Br;
C₆-C₁₂ alkylcycloalkyl;
C₆-C₁₂ alkylcycloalkyl containing at least one hetero atom selected from the group consisting of 0 forming a hydroxyl-, carbonyl-, ether- or ester-group, N, Si, Cl, and Br;
C₆-C₁₂ cycloalkylalkyl;
C₆-C₁₂ cycloalkylalkyl containing at least one hetero atom selected from the group consisting of O forming a hydroxyl-, carbonyl-, ether- or ester-group, N, Si, Cl, and Br;
C₂-C₁₀ alkenyl;
C₂-C₁₀ alkenyl containing at least one hetero atom selected from the group consisting of O forming a hydroxyl-, carbonyl-, ether- or ester-group, N, Si, Cl, and Br;
C₂-C₁₀ alkinyl;
C₂-C₁₀ alkinyl containing at least one hetero atom selected from the group consisting of O forming a hydroxyl-, carbonyl-, ether- or ester-group, N, Si, Cl, and Br;
C₆-C₁₀ aryl;
C₆-C₁₀ aryl substituted with at least one substituent selected from the group consisting of C₁-C₁₅ alkyl and C₁-C₁₅ alkoxy;
C₅-C₁₀ heteroaryl;
C₇-C₁₀ alkylaryl; and
C₇-C₁₀ alkylaryl substituted with at least one substituent selected from the group consisting of C₁-C₁₁ alkyl, C₁-C₁₅ alkoxy, amino, C₁-C₁₅ alkylamino, and C₂-C₃₀ dialkylamino.

3. The method according to claim 1 wherein in the compound of formula 1: A is selected from the group consisting of H, C₁-C₉ alkyl, —COOC₂H₅, —C(O)CH₃ and —COOCH₂Ph.

4. The method according to claim 1 wherein in the compound of formula 1:
A is —CR⁹R¹⁰R¹¹ wherein R⁹, R¹⁰, and R¹¹ form together with the carbon atom to which they are attached a C₆-C₁₀ aryl group with at least one substitutent selected from hydroxyl, C₁-C₅ alkoxy, acetyl and amino; or
A is —CR⁹R¹⁰R¹¹ wherein R⁹, R¹⁰, and R¹¹ form together with the carbon atom to which they are attached a C₅-C₈ heteroaryl.

5. The method according to claim 1 wherein in the compound of formula 1:
A is —CR⁹R¹⁰R¹¹ and R⁹, R¹⁰, and R¹¹ form together with the carbon atom to which they are attached pyridinyl.

6. The method according to claim 1 wherein in the compound of formula 1 B is H, hydroxyl, —O—C₂H₅, —O—(CH₂)₂—O—(CH₂)₂—O—C₂H₅, —O—C(O)CH₃ or —O—C(O)C₂H₅.

7. The method according to claim 1 wherein the compound of a formula 1 is selected from the group consisting of:
2-(3,7-dimethyl-octa-2,6-dienylidene)-malonic acid diethyl ester;
3-octylidene-pentane-2,4-dione;
2-pyridin-2-ylmethylene-malonic acid diethyl ester;
2-octylidene-malonic acid dimethyl ester;
2-ethoxycarbonyl-but-2-enedioic acid diethyl ester;
2-acetyl-pent-2-enoic acid ethyl ester;
2-octylidene-malonic acid diethyl ester;
2-decylidene-malonic acid diethyl ester;
2-acetyl-dec-2-enoic acid ethyl ester;
2-(2-hydroxy-benzylidene)-1-phenyl-butane-1,3-dione;
2-(2-hydroxy-benzylidene)-malonic acid diethyl ester;
3-(2-hydroxy-benzylidene)-pentane-2,4-dione;
3-(2-hydroxy-4-methoxy-benzylidene)-pentane-2,4-dione;
2-(2-hydroxy-benzylidene)-3-oxo-butyric acid ethyl ester;
2-ethoxycarbonyloxymethylene-malonic acid diethyl ester;
2-acetoxymethylene-malonic acid diethyl ester;
2-benzoyl-dec-2-enoic acid ethyl ester;
2-acetyl-5,9-dimethyl-deca-2,8-dienoic acid ethyl ester;
2-acetyl-dec-2-enoic acid 2-(2-methoxy-ethoxy)-ethyl ester;
1-(4-tert-butyl-phenyl)-3-(4-methoxy-phenyl)-2-octylidene-propane-1,3-dione;
2-octylidene-malonic acid dibenzyl ester;
2-carbamoyl-dec-2-enoic acid methyl ester;
3-(3-ethoxy-4-hydroxy-benzylidene)-pentane-2,4-dione;
2-acetyl-3-hydroxy-succinic acid 4-ethyl ester 1-(2-methoxy-ethyl) ester;
3-acetyl-2-hydroxy-4-oxo-pentanoic acid ethyl ester;
2-acetyl-3-hydroxy-succinic acid diethyl ester;
2-benzoyl-3-hydroxy-succinic acid diethyl ester;
2-butyryl-3-hydroxy-succinic acid diethyl ester;
2-acetyl-3-hydroxy-succinic acid 1-tert-butyl ester 4-ethyl ester;
2-(2,2-dimethyl-propionyl)-3-hydroxy-succinic acid 4-ethyl ester 1-methyl ester;
2-acetyl-3-hydroxy-succinic acid 4-ethyl ester 1-(2-methoxy-ethyl) ester;
2-butyryl-but-2-enedioic acid diethyl ester;
2-acetyl-but-2-enedioic acid 1-benzyl ester 4-ethyl ester;
2-acetyl-but-2-enedioic acid 4-ethyl ester 1-(2-methoxy-ethyl) ester;
3-carbamoyl-4-oxo-pent-2-enoic acid ethyl ester;
2-(2,2-dimethyl-propionyl)-but-2-enedioic acid 4-ethyl ester 1-methyl ester;
2-pent-4-ynoyl-but-2-enedioic acid 4-ethyl ester 1-methyl ester;
2-acetyl-3-methoxy-succinic acid diethyl ester;
3-benzoyl-2-ethoxy-4-oxo-pentanoic acid ethyl ester;
2-butyryl-3-ethoxy-succinic acid diethyl ester;
2-benzoyl-3-ethoxy-succinic acid diethyl ester;
2-acetyl-3-[2-(2-ethoxy-ethoxy)-ethoxy]-succinic acid diethyl ester;
3-acetyl-2-ethoxy-4-oxo-pentanoic acid ethyl ester;
2-propylidene-1-(pyridin-4-yl)butane-1,3-dione;
2-ethylidene-1-(1H-imidazol-5-yl)butane-1,3-dione;
3-(2-hydroxy-5-nitrobenzylidene)pentane-2,4-dione;
3-(2-hydroxy-5-acetylbenzylidene)pentane-2,4-dione;
ethyl-2-hydroxy-4-(1H-imidazol-4-yl)-3-acetyl-4-oxobutanoate;
4-ethyl 1-(2-(trimethylsilyl)ethyl)-2-acetylmaleate;
ethyl-4-cyclohexyl-4-oxo-3-acetyl-but-2-enoate;
ethyl 3-((trimethylsilyl)methylcarbamoyl)-4-oxopent-2-enoate;
ethyl-4-(4-methylcyclohexyl)-4-oxo-3-acetyl-but-2-enoate;
1-(2-(dimethylamino)ethyl) 4-ethyl 2-acetylmaleate;
benzyl 3-acetyl-4-oxopent-2-enoate; and
4-acetyl-9-methyldeca-3,8-diene-2,5-dione.

8. A consumer product comprising a compound a compound according to formula 1 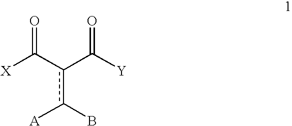
wherein
X and Y are independently a residue selected from the group consisting of —CR¹R²R³, wherein
R¹, R², and R³ are independently H, a hydrocarbon residue, or a hydrocarbon residue containing at least one hetero atom selected from the list consisting of oxygen forming a hydroxyl-, carbonyl-, ether- or ester-group, nitrogen, silicon, chlorine and bromine;
—NR⁴R⁵, wherein
R⁴ and R⁵ are independently H, a hydrocarbon residue; or
R⁴ and R⁵ form together with the nitrogen atom to which they are attached a 3, 5 or 6-membered ring; or R⁴ and R⁵ are a hydrocarbon residue containing at least one hetero atom selected from the list consisting of oxygen forming a hydroxyl-, carbonyl-, ether- or ester-group, nitrogen, silicon, chlorine and bromine;
and
—OR⁶, wherein
R⁶ is a hydrocarbon residue; or
R⁶ is a hydrocarbon residue containing at least one hetero atom selected from the list consisting of oxygen forming a hydroxyl-, carbonyl-, ether- or ester-group, nitrogen, silicon, chlorine and bromine;
A is H; —COOR⁷, wherein
R⁷ is a hydrocarbon residue; or
R⁷ is a hydrocarbon residue containing at least one hetero atom selected from the group consisting of oxygen forming a hydroxyl-, carbonyl-, ether- or ester-group, nitrogen, silicon, chlorine and bromine;
—C(O)R⁸, wherein
R⁸ is a hydrocarbon residue; or
R⁸ is a hydrocarbon residue containing at least one hetero atom selected from the group consisting of oxygen forming a hydroxyl-, carbonyl-, ether- or ester-group, nitrogen, silicon, chlorine and bromine;
or
—CR⁹R¹⁰R¹¹, wherein
R⁹, R¹⁰ and R¹¹ are independently H, a hydrocarbon residue, or a hydrocarbon residue containing at least one hetero atom selected from the group consisting of oxygen forming a hydroxyl-, carbonyl-, ether- or ester-group, nitrogen, silicon, chlorine and bromine;
B is H; —CR¹²R¹³R¹⁴, wherein
R¹², R¹³ and R¹⁴ are independently H, or a hydrocarbon residue; or
R¹², R¹³ and R¹⁴ are independently a hydrocarbon residue containing at least one hetero atom selected from the group consisting of oxygen forming a hydroxyl-, carbonyl-, ether- or ester-group, nitrogen, silicon, chlorine and bromine;
or
—OC(O)R¹⁸, wherein
R¹⁸ is H, or a hydrocarbon residue;
and the dotted line together with the carbon-carbon bond represents a double bond; or
B is —NR¹⁶R¹⁷, wherein
R¹⁶ and R¹⁷ are independently H, or a hydrocarbon residue; or
R¹⁶ and R¹⁷ are independently a hydrocarbon residue containing at least one hetero atom selected from the group consisting of oxygen forming a hydroxyl-, carbonyl-, ether- or ester-group nitrogen, silicon, chlorine and bromine; or
—OR¹⁵, wherein
R¹⁵ is H, or a hydrocarbon residue; or
R¹⁵ is a hydrocarbon residue containing at least one hetero atom selected from the group consisting of oxygen forming a hydroxyl-, carbonyl-, ether- or ester-group, nitrogen, silicon, chlorine and bromine;
and the dotted line together with the carbon-carbon bond represents a single bond or a double bond;
with the proviso that A and B are not hydrogen at the same time.

9. A consumer product according to claim 8, wherein the consumer product is selected from the group consisting of cosmetic products, home care products and fabric care products

10. A consumer product according to claim 8 comprising about 0.01 to about 20 weight % of a compound of formula 1.

11. A method of imparting a malodour neutralizing effect to a substrate comprising contacting the substrate with a consumer product according to claim 8.

12. A method according to claim 11 wherein the substrate is selected from the group consisting of fabrics, hard surfaces, skin and hair.

13. A process for dispersing a consumer product into a space comprising:
(a) incorporating into a consumer product a compound of formula 1 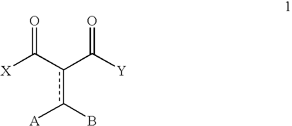
wherein
X and Y are independently a residue selected from the group consisting of —CR¹R²R³, wherein
R¹, R², and R³ are independently H, a hydrocarbon residue, or a hydrocarbon residue containing at least one hetero atom selected from the list consisting of oxygen forming a hydroxyl-, carbonyl-, ether- or ester-group, nitrogen, silicon, chlorine and bromine;
—NR⁴R⁵, wherein
R⁴ and R⁵ are independently H, a hydrocarbon residue; or
R⁴ and R⁵ form together with the nitrogen atom to which they are attached a 3, 5 or 6-membered ring; or R⁴ and R⁵ are a hydrocarbon residue containing at least one hetero atom selected from the list consisting of oxygen forming a hydroxyl-, carbonyl-, ether- or ester-group, nitrogen, silicon, chlorine and bromine;
and
—OR⁶, wherein
R⁶ is a hydrocarbon residue; or
R⁶ is a hydrocarbon residue containing at least one hetero atom selected from the list consisting of oxygen forming a hydroxyl-, carbonyl-, ether- or ester-group, nitrogen, silicon, chlorine and bromine;
A is H; —COOR⁷, wherein
R⁷ is a hydrocarbon residue; or
R⁷ is a hydrocarbon residue containing at least one hetero atom selected from the group consisting of oxygen forming a hydroxyl-, carbonyl-, ether- or ester-group, nitrogen, silicon, chlorine and bromine;
—C(O)R⁸, wherein
R⁸ is a hydrocarbon residue; or
R⁸ is a hydrocarbon residue containing at least one hetero atom selected from the group consisting of oxygen forming a hydroxyl-, carbonyl-, ether- or ester-group, nitrogen, silicon, chlorine and bromine;
or
—CR⁹R¹⁰R¹¹, wherein
R⁹, R¹⁰ and R¹¹ are independently H, a hydrocarbon residue, or a hydrocarbon residue containing at least one hetero atom selected from the group consisting of oxygen forming a hydroxyl-, carbonyl-, ether- or ester-group, nitrogen, silicon, chlorine and bromine;
B is H; —CR¹²R¹³R¹⁴, wherein
R¹², R¹³ and R¹⁴ are independently H, or a hydrocarbon residue; or
R¹², R¹³ and R¹⁴ are independently a hydrocarbon residue containing at least one hetero atom selected from the group consisting of oxygen forming a hydroxyl-, carbonyl-, ether- or ester-group, nitrogen, silicon, chlorine and bromine;
or
—OC(O)R¹⁸, wherein
R¹⁸ is H, or a hydrocarbon residue;
and the dotted line together with the carbon-carbon bond represents a double bond; or
B is —NR¹⁶R¹⁷, wherein
R¹⁶ and R¹⁷ are independently H, or a hydrocarbon residue; or
R¹⁶ and R¹⁷ are independently a hydrocarbon residue containing at least one hetero atom selected from the group consisting of oxygen forming a hydroxyl-, carbonyl-, ether- or ester-group nitrogen, silicon, chlorine and bromine; or
—OR¹⁵, wherein
R¹⁵ is H, or a hydrocarbon residue; or
R¹⁵ is a hydrocarbon residue containing at least one hetero atom selected from the group consisting of oxygen forming a hydroxyl-, carbonyl-, ether- or ester-group, nitrogen, silicon, chlorine and bromine;
and the dotted line together with the carbon-carbon bond represents a single bond or a double bond;
with the proviso that A and B are not hydrogen at the same time; and
(b) dispersing an effective amount of the consumer product into the space.

14. A process according to claim 13 wherein the consumer product is selected from the group of home care products.

15. A compound of formula 1a 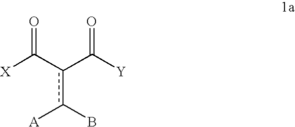
wherein
X and Y are independently a residue selected from the group consisting of —CR¹R²R³, wherein
R¹, R², and R³ are independently H, a hydrocarbon residue, or a hydrocarbon residue containing at least one hetero atom selected from the list consisting of oxygen forming a hydroxyl-, carbonyl-, ether- or ester-group, nitrogen, silicon, chlorine and bromine;
—NR⁴R⁵, wherein
R⁴ and R⁵ are independently H, a hydrocarbon residue; or
R⁴ and R⁵ form together with the nitrogen atom to which they are attached a 3, 5 or 6-membered ring; or R⁴ and R⁵ are a hydrocarbon residue containing at least one hetero atom selected from the list consisting of oxygen forming a hydroxyl-, carbonyl-, ether- or ester-group, nitrogen, silicon, chlorine and bromine;
and
—OR⁶, wherein
R⁶ is a hydrocarbon residue; or
R⁶ is a hydrocarbon residue containing at least one hetero atom selected from the list consisting of oxygen forming a hydroxyl-, carbonyl-, ether- or ester-group, nitrogen, silicon, chlorine and bromine;
A is —COOR⁷, wherein
R⁷ is a hydrocarbon residue; or
R⁷ is a hydrocarbon residue containing at least one hetero atom selected from the group consisting of oxygen forming a hydroxyl-, carbonyl-, ether- or ester-group, nitrogen, silicon, chlorine and bromine; or
A is —C(O)R⁸, wherein
R⁸ is a hydrocarbon residue; or
R⁸ is a hydrocarbon residue containing at least one hetero atom selected from the group consisting of oxygen forming a hydroxyl-, carbonyl-, ether- or ester-group, nitrogen, silicon, chlorine and bromine;
B is —NR¹⁶R¹⁷, wherein
R¹⁶ and R¹⁷ are independently H, or a hydrocarbon residue; or
R¹⁶ and R¹⁷ are independently a hydrocarbon residue containing at least one hetero atom selected from the group consisting of oxygen forming a hydroxyl-, carbonyl-, ether- or ester-group, nitrogen, silicon, chlorine and bromine; or
B is —OR¹⁵, wherein
R¹⁵ is H, or a hydrocarbon residue; or
R¹⁵ is a hydrocarbon residue containing at least one hetero atom selected from the group consisting of oxygen forming a hydroxyl-, carbonyl-, ether- or ester-group, nitrogen, silicon, chlorine and bromine;
and the dotted line together with the carbon-carbon bond represents a single bond;
with the proviso that
if X═Y is ethoxy, B is not methoxy; and
if X is ethoxy and Y is methyl, B is not methoxy or ethoxy.

16. A compound according to claim 15 wherein A is —COOC₂H₅.

17. A compound according to claim 15 wherein B is hydroxyl, or —O—C₂H₅.

18. A compound according to claim 15 selected from the group consisting of:
3-acetyl-2-hydroxy-4-oxo-pentanoic acid ethyl ester;
2-acetyl-3-hydroxy-succinic acid diethyl ester;
2-benzoyl-3-hydroxy-succinic acid diethyl ester;
2-acetyl-3-hydroxy-succinic acid 4-ethyl ester 1-(2-methoxy-ethyl) ester;
3-benzoyl-2-ethoxy-4-oxo-pentanoic acid ethyl ester;
2-butyryl-3-ethoxy-succinic acid diethyl ester; and
2-benzoyl-3-ethoxy-succinic acid diethyl ester.

19. A compound of formula 1b 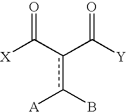
wherein
X and Y are independently a residue selected from the group consisting of —CR¹R²R³, wherein
R¹, R², and R³ are independently H, a hydrocarbon residue, or a hydrocarbon residue containing at least one hetero atom selected from the list consisting of oxygen forming a hydroxyl-, carbonyl-, ether- or ester-group, nitrogen, silicon, chlorine and bromine;
—NR⁴R⁵, wherein
R⁴ and R⁵ are independently H, a hydrocarbon residue; or
R⁴ and R⁵ form together with the nitrogen atom to which they are attached a 3, 5 or 6-membered ring; or R⁴ and R⁵ are a hydrocarbon residue containing at least one hetero atom selected from the list consisting of oxygen forming a hydroxyl-, carbonyl-, ether- or ester-group, nitrogen, silicon, chlorine and bromine;
and
—OR⁶, wherein
R⁶ is a hydrocarbon residue; or
R⁶ is a hydrocarbon residue containing at least one hetero atom selected from the list consisting of oxygen forming a hydroxyl-, carbonyl-, ether- or ester-group, nitrogen, silicon, chlorine and bromine;
A is selected from the group consisting of n-heptyl, —COOC₂H₅, and —CH₂—CH(CH)₃—(CH₂)₂—CH═CH(CH₃)₂;
B is hydrogen;
and the dotted line together with the carbon-carbon bond represents a double bond;
with the proviso that
if X═Y is methyl or methoxy, A is not —COOC₂H₅;
if X═Y is methyl, methoxy or ethoxy, A is not n-heptyl;
if X is methyl and Y is ethoxy, A is not n-heptyl.

20. A compound according to claim 19 selected from the group consisting of:
2-benzoyl-dec-2-enoic acid ethyl ester;
2-acetyl-5,9-dimethyl-deca-2,8-dienoic acid ethyl ester;
2-acetyl-dec-2-enoic acid 2-(2-methoxy-ethoxy)-ethyl ester;
1-(4-tert-butyl-phenyl)-3-(4-methoxy-phenyl)-2-octylidene-propane-1,3-dione;
2-octylidene-malonic acid dibenzyl ester;
2-carbamoyl-dec-2-enoic acid methyl ester;
2-butyryl-but-2-enedioic acid diethyl ester;
2-acetyl-but-2-enedioic acid 1-benzyl ester 4-ethyl ester;
2-acetyl-but-2-enedioic acid 4-ethyl ester 1-(2-methoxy-ethyl) ester;
3-carbamoyl-4-oxo-pent-2-enoic acid ethyl ester;
2-(2,2-dimethyl-propionyl)-but-2-enedioic acid 4-ethyl ester 1-methyl ester;
2-pent-4-ynoyl-but-2-enedioic acid 4-ethyl ester 1-methyl ester;
4-ethyl 1-(2-(trimethylsilyl)ethyl)-2-acetylmaleate;
ethyl-4-cyclohexyl-4-oxo-3-acetyl-but-2-enoate;
ethyl 3-((trimethylsilyl)methylcarbamoyl)-4-oxopent-2-enoate;
ethyl-4-(4-methylcyclohexyl)-4-oxo-3-acetyl-but-2-enoate; and
1-(2-(dimethylamino)ethyl) 4-ethyl 2-acetylmaleate.
